SEQUENCE LISTING (17 sequences)

SEQ ID NO: 1 - protein, 380 aa, Homo sapiens
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 2 - protein, 6 aa, Homo sapiens
  UNSURE (1)..(1): Xaa in position 1 is methionine or nor-leucin
  UNSURE (4)..(4): Xaa in position 4 is cysteine or alanine
XDGXEL

SEQ ID NO: 3 - protein, 6 aa, Homo sapiens
MDGCEL

SEQ ID NO: 4 - protein, 7 aa, Homo sapiens
GMDGCEL

SEQ ID NO: 5 - protein, 8 aa, Homo sapiens
EGMDGCEL

SEQ ID NO: 6 - protein, 9 aa, Homo sapiens
SEGMDGCEL

SEQ ID NO: 7 - protein, 10 aa, Homo sapiens
TSEGMDGCEL

SEQ ID NO: 8 - protein, 11 aa, Homo sapiens
KTSEGMDGCEL

SEQ ID NO: 9 - protein, 12 aa, Homo sapiens
NKTSEGMDGCEL

SEQ ID NO: 10 - protein, 13 aa, Homo sapiens
CNKTSEGMDGCEL

SEQ ID NO: 11 - protein, 14 aa, Homo sapiens
LCNKTSEGMDGCEL

SEQ ID NO: 12 - protein, 15 aa, Homo sapiens
RLCNKTSEGMDGCEL

SEQ ID NO: 13 - protein, 16 aa, Homo sapiens
GRLCNKTSEGMDGCEL

SEQ ID NO: 14 - protein, 17 aa, Homo sapiens
QGRLCNKTSEGMDGCEL

SEQ ID NO: 15 - protein, 18 aa, Homo sapiens
TQGRLCNKTSEGMDGCEL

SEQ ID NO: 16 - protein, 19 aa, Homo sapiens
GTQGRLCNKTSEGMDGCEL

SEQ ID NO: 17 - protein, 20 aa, Homo sapiens
LGTQGRLCNKTSEGMDGCEL

(19) United States
(12) Patent Application Publication
(10) Pub. No.: US 2022/0202901 A1
(43) Pub. Date: Jun. 30, 2022

(54) PEPTIDES IN COMBINATION WITH IMMUNE CHECKPOINT INHIBITORS FOR USE IN TREATMENT OF CANCER

(71) Applicant: WNTRESEARCH AB, Malmö (SE)

(72) Inventors: Peter Johannes HOLST, København K (DK); Lena AXELSSON, Malmö (SE); Tommy ANDERSSON, Malmö (SE)

(21) Appl. No.: 17/604,356

(22) PCT Filed: Apr. 16, 2020
(86) PCT No.: PCT/EP2020/060750

§ 371 Date: Oct. 15, 2021

(30) Foreign Application Priority Data
Apr. 16, 2019 (EP) 19169388.6

(51) Int. Cl. A61K 38/18; A61K 39/395; A61P 35/00

(52) U.S. Cl. CPC A61K 38/18; A61P 35/00; A61K 39/3955

(57) ABSTRACT

A WNT5A peptide or derivatives thereof in combination with one or more checkpoint inhibitors for use in treatment of cancer in a subject in need thereof. Furthermore, WNT5A peptides or derivatives thereof may be used in treatment of cancer in a subject, wherein the subject is responsive to immune checkpoint inhibitors.

### TECHNICAL FIELD

A WNT5A peptide or derivatives thereof for use in treatment of cancer in a subject responsive to check point inhibitors.

### BACKGROUND ART

The link between the immune system and cancer has long been appreciated. The immune system acts to defend and protect an individual by detecting “non-self” and overexpressed antigens from pathogens or infected/malignant cells; target and destroy the pathogen or infected/malignant cells while protecting the host; and it develops immunological memory via the adaptive immune responses for subsequent defense mechanisms.

Checkpoint inhibitors (ICIs) are a type of drug that block so-called immune checkpoints. Immune checkpoint ligands appear on the surface of tumor cells and immune dampening immune cells, whereas the cognate molecules appear on the surface of tumor reactive immune system cells such as T cells and natural killer cells. These molecules help to dampen the immune responses and prevent over activation of the immune system. When cancer specific T cells are not inhibited by immune checkpoints, the T cells will kill cancer cells. Examples of immune checkpoints found on T cells or cancer cells include the molecule PD-1 and its ligand PD-L1, and, CTLA-4, which competes with the co-stimulatory molecule CD28 for binding to B7-1/B7-2.

Since many of the immune checkpoints are regulated by interactions between specific molecules and ligand pairs, monoclonal antibodies or other agents can be used to block this interaction and prevent immunosuppression. Immune checkpoint inhibitors are thus used in the treatment of cancer by their ability to block the checkpoint protein molecules causing inhibition of the T-cells. Examples of currently known ICIs block cytotoxic T lymphocyte antigen 4 (CTLA-4; i.e., ipilimumab), programmed death 1 (PD-1; i.e., nivolumab, pembrolizumab, cemiplimab), or programmed death ligand 1 (PD-L1; i.e., atezolizumab, avelumab, durvalumab).

Some proteins involved in immune checkpoints, using B7-1/B7-2 as an example, help tell T cells to become active by signalling through the costimulatory receptor CD28, for example when an infection is present. However, if T cells are active for too long, or react to targets inappropriately, they can start to destroy healthy cells and tissues and the immune checkpoint molecule, CTLA-4, blocks the interaction between CD28 and B7-1/B7-2.

Some cancer cells produce high levels of checkpoint protein ligands causing the T cells to switch off, when they should ideally be attacking the cancer cells. So the cancer cells are pushing a stop button on the immune system. This is the category of cancer patients that will tend to respond to ICI therapy. Response rates to treatment with checkpoint inhibitors remain relatively low, ranging from 15 to 40% depending on cancer type.

Primary and acquired resistance are key clinical barriers to further improving patient outcome for some types of cancers, and the known mechanisms underlying each involve various components of the cancer immune cycle, and interactions between multiple signalling molecules and pathways. Due to this complexity, current knowledge on resistance mechanisms is still incomplete. Overcoming therapy resistance requires a thorough understanding of the mechanisms underlying immune evasion by tumors.

Attempts have been made to provide combination therapies. For example, radiotherapy in combination with checkpoint inhibitors as well as treatment with a combination of checkpoint inhibitors have been tested. One drawback with such therapies is that combination therapy may be more toxic to the patient than a single treatment.

On this background it is an object of the present invention to provide improved therapies that are i.a. for use in treatment of cancer such therapies being rather non-toxic or less toxic to the patient and comprising administering a therapeutic agent that is more compliant to the patients. It is further an object of the present invention to improve the effect of checkpoint inhibitors.

### SUMMARY OF THE INVENTION

Thus, according to a first aspect of the present invention, there is provided a WNT5A peptide or derivatives thereof in combination with one or more checkpoint inhibitors for use in treatment of cancer in a subject in need thereof, the WNT5A peptide comprising X_{A}DGX_{B}EL (SEQ. ID. NO. 2), or a formylated derivative thereof, wherein X_{A} is methionine (M) or norleucine, X_{B} is cysteine (C) or alanine (A), wherein the total length of the peptide is equal to or less than 50 amino acids, wherein said peptide and said checkpoint inhibitor are either combined or separate and/or are administered either simultaneously or sequentially. The amino acid residues of said WNT5A peptide, except glycine, may be either in the L- or D-stereoisomeric form.

It has been found that WNT5A peptides and derivatives of the form outlined above in combination with one or more checkpoint inhibitors can be used to reduce tumour growth and hence in the treatment of cancer in certain subjects. It is currently believed that the WNT5A peptides cause a lower expression level of checkpoints on the cancer cells. The lower expression of the check points means that a lower amount of check point inhibitor will be necessary or that a higher efficacy may be observed. The mechanism underlying this, however, is not at present well understood.

In some embodiments, said subject is defined as being sensitive or responsive to immune checkpoint inhibitors. Responsiveness to immune check point inhibitors is to be understood as subjects having checkpoints, preferably CTLA-4, PD-L1 and/or CD47 expression by any of tumour cells or infiltrating immune cells as well as their respective counterparts.

In some embodiments, the total length of the WNT5A peptide is equal to or less than 20 amino acids.

In some embodiments at least one checkpoint inhibitor is an inhibitor of an immune checkpoint selected from the group consisting of but not limited to CTLA-4, PD-L1 and CD47, most preferred CD47. In further embodiments the check point inhibitor is an antibody such as anti-CTLA4-antibody, anti-PD-L1-antibody and/or anti-CD47-antibody. It is contemplated that the check point inhibitor may be the anti-CTLA-4 antibody ipilimumab or tremelimumab, PD-1 blocking antibodies such as nivolumab or anti-PD-L1-antibody antibody is atezolizumab, avelumab, durvalumab or pembrolizumab, or a combination of antibodies thereof.

Ipilimumab is the International non-proprietary name (INN) or common name for is a fully human anti-CTLA-4 monoclonal antibody (IgG1κ) and is currently produced in mammalian cells such as in Chinese hamster ovary cells by recombinant DNA technology. The trade name for ipilimumab is Yervoy®

Tremelimumab is a fully human monoclonal antibody against CTLA-4.

Nivolumab is the International non-proprietary name (INN) or common name for a human immunoglobulin G4 (IgG4) monoclonal antibody (HuMAb), binds to the programmed death-1 (PD-1) receptor and blocks the interaction with programmed death-ligand 1 (PDL1) and programmed death-ligand 2 (PD-L2) and is currently produced in mammalian cells such as in Chinese hamster ovary cells by recombinant DNA technology. The trade name for nivolumab is Opdivo®.

Atezolizumab is the International non-proprietary name (INN) or common name for an Fc-engineered, humanised IgG1 anti-programmed death-ligand 1 (PD-L1) monoclonal antibody and is currently produced in mammalian cells such as in Chinese hamster ovary cells by recombinant DNA technology. The trade name for atezolizumab is Tecentriq®.

Avelumab is the International non-proprietary name (INN) or common name for a human monoclonal IgG1 antibody directed against the immunomodulatory cell surface ligand protein PD-L1 and is currently produced in mammalian cells such as in Chinese hamster ovary cells by recombinant DNA technology. The trade name for avelumab is Bavencio®.

Durvalumab is the International non-proprietary name (INN) or common name for an antineoplastic monoclonal antibody that potentiates T-cell response, including antitumour response, through blockade of PD-L1 binding to PD-1 and is currently produced in mammalian cells such in Chinese hamster ovary cells by recombinant DNA technology. The trade name for durvalumab is Imfinzi®.

Pembrolizumab is the International non-proprietary name (INN) or common name for is a humanised monoclonal anti-programmed cell death-1 (PD-1) antibody (IgG4/kappa isotype with a stabilising sequence alteration in the Fc region) and is currently produced in mammalian cells such in Chinese hamster ovary cells by recombinant DNA technology. The trade name for pembrolizumab is Keytruda®.

The WNT5A peptide or derivatives thereof in combination with one or more checkpoint inhibitors for use in the treatment of cancer in a subject in need thereof allows for the dosage of checkpoint inhibitor used to be reduced as compared to the dosage used when not administering WNT5A simultaneously or sequentially. This synergistic effect of the invention is particularly beneficial in terms of patient compliance, as the side effects as a consequence hereof are reduced.

A low expression of WNT5A in breast, colon and prostate cancer tumours have been correlated with an increased number of disease recurrences and a shortened survival time of the patient (Mehdawi LM1, Prasad CP1, Ehrnström R2, Andersson T1, Sjölander A, Non-canonical WNT5A signaling up-regulates the expression of the tumor suppressor 15-PGDH and induces differentiation of colon cancer cells. Mol Oncol. 2016 November; 10(9):1415-1429).

Non-canonical WNT5A signaling up-regulates the expression of the tumor suppressor 15-PGDH and induces differentiation of colon cancer cells.
WNT5A is known to inhibit migration of cells of these cancer types in the body, and the addition of recombinant WNT5A has been shown to impair the migration of these cells. Preferably the cancer is colon cancer such as colorectal cancer or breast cancer.

The subject diagnosed with cancer may show upregulated tumour expression of one or more immune checkpoints selected from the group consisting of CTLA-4, PD-L1 and CD47 as compared to normal cells in the subject.

The WNT5A peptide is suitably administered together with an anti-PD-L1-antibody and/or an anti-CTLA4-antibody and wherein the subject in need thereof has an upregulated tumour expression of CTLA-4 and/or PD-L1.

In the context of the invention upregulated expression means that a cell increase the quantity of a cellular component, such as CTLA-4 and/or PD-L1, in response to an external stimulus such as treatment with WNT5A peptide or Foxy-5. The complementary process that involves decreasing of such components is called downregulation.

The WNT5A peptide and derivatives used in the treatment of cancer in a subject in need thereof, wherein at least one peptide is selected from the group consisting of:

(SEQ. ID. NO. 3)
MDGCEL,
(SEQ. ID. NO. 4)
GMDGCEL,
(SEQ. ID. NO. 5)
EGMDGCEL,
(SEQ. ID. NO. 6)
SEGMDGCEL,
(SEQ. ID. NO. 7)
TSEGMDGCEL,
(SEQ. ID. NO. 8)
KTSEGMDGCEL,
(SEQ. ID. NO. 9)
NKTSEGMDGCEL,
(SEQ. ID. NO. 10)
CNKTSEGMDGCEL,
(SEQ. ID. NO. 11)
LCNKTSEGMDGCEL,
(SEQ. ID. NO. 12)
RLCNKTSEGMDGCEL,
(SEQ. ID. NO. 13)
GRLCNKTSEGMDGCEL,
(SEQ. ID. NO. 14)
QGRLCNKTSEGMDGCEL,
(SEQ. ID. NO. 15)
TQGRLCNKTSEGMDGCEL,
(SEQ. ID. NO. 16)
GTQGRLCNKTSEGMDGCEL,
and
(SEQ. ID. NO. 17)
LGTQGRLCNKTSEGMDGCEL.

In one embodiment, the WNT5A peptide in combination with one or more checkpoint inhibitors for use in the treatment of cancer in a subject in need thereof, is hexapeptide MDGCEL or a formylated derivative thereof. The formylated derivative thereof is sometimes referred to as Foxy-5 herein.

In another aspect, the WNT5A peptide or derivatives thereof is used in treatment of cancer in a subject, said subject being defined as responsive to immune checkpoint inhibitors, the WNT5A peptide comprising X_{A}DGX_{B}EL (SEQ. ID. NO. 2), or a formylated derivative thereof, wherein X_{A} is methionine (M) or norleucine, X_{B} is cysteine (C) or alanine (A), wherein the total length of the peptide is equal to or less than 50 amino acids.

In some embodiments the subject diagnosed with cancer has an upregulated tumour expression of one or more immune checkpoints selected from the group consisting of CTLA-4, PD-L1 and CD47.

### BRIEF DESCRIPTION OF THE FIGURES

The invention will be described in more detail below by means of non-limiting example of embodiments and with reference to the figures, in which:

FIG. 1 shows the effect on 4T1 breast cancer cell specific T cell responses with FOXY-5 and ICI co-treatment. IFNγ spot forming cells were counted after MuLV gp70 derived peptide stimulation.

FIG. 2a-2d shows tumour volume after subcutaneous implantation of 4T1breast cancer cells in BALB-C mice with and without ICI and Foxy-5 treatment.

FIG. 3 shows the effect of Foxy-5 on CD47 expression in mouse triple-negative 4T1 breast cancer cells (by Western blot and subsequent densitometry).

FIG. 4 shows the effect of INFγ and Foxy-5 on PD-L1 expression in mouse triple-negative 4T1 breast cancer cells (by Western blot and subsequent densitometry).

### DETAILED DESCRIPTION

The WNT (Wingless-related integration site) protein family contains highly conserved proteins that play a role in embryonic development such as body axis patterning, cell proliferation and migration. The WNT signalling pathways are either canonical or non-canonical and they primarily trigger the regulation of gene transcription and increased proliferation via canonical signalling or regulation of several non-proliferative functions via activation of different non-canonical signalling pathways in the cells. The WNT proteins are further involved in tissue regeneration in adult bone marrow, skin and intestine. Genetic mutation in the WNT signalling pathway may cause breast cancer, prostate cancer glioblastoma, type II diabetes and other diseases.

The canonical WNT pathway activates β-catenin and is integral in regulating self-renewal of normal stem cells and the subversion of the canonical WNT signalling has been implicated in tumourigenesis. In contrast, non-canonical WNT signalling is characterized by an absence of an increase in β-catenin signalling and has been studied for its role in embryonic patterning, gastrulation, and organogenesis. Moreover, non-canonical WNT is proposed to antagonize canonical signalling. WNT5A is an example of a non-canonical WNT ligand. WNT5A is tumour-suppressive in acute myelogenous leukemia (AML), colon cancer including colo-rectal cancer, breast and prostate cancer, and ovarian carcinoma.

WNT5A is a protein expressed by many normal cells in the body. WNT5A is secreted from the cells and exerts its action on the same or neighbouring cells by binding to and activating a receptor complex primarily involving a Frizzled receptor. The WNT5A protein is known to activate different Frizzled receptors. Upon activation of the Frizzled 5 receptor a series of signalling events inside of the cells are activated, where one of the first events, is generation of short-lived increase in calcium inside of the cell, a so called calcium-signal. The calcium-signal in turn triggers a series of forthcoming signalling events leading to a change in the functions of the cells, such as adhesion and migration. Thus, activating such a Frizzled receptor leads to signalling events inside the cell, resulting in increased adherence of the cell to its neighbouring cells and its adhesion to the surrounding connective tis-sue resulting in decreased ability of the tumour cell to migrate to structures in the vicinity, such as lymph nodes and blood vessels. In healthy breast epithelial cells for example, WNT5A is highly expressed and secures a firm adherence between cells and to the surrounding basement membrane and thereby restricts migration of the cells.

In order to reconstitute WNT5A signalling in cancer tissue that lack an endogenous expression of WNT5A, a small peptide, i.e. equal to or less than 20 amino acids derived from the amino acid sequence of the WNT5A molecule has been developed and then additionally modified. An example of such a peptide is Foxy-5, which is a true WNT5A agonist in that it triggers the same signalling events and functional responses as WNT5A and in comparison, with WNT5A it is a much simpler molecule and it can be administered systemically and still reach the tumour tissue. Thus, the term signalling properties, as used herein, means binding of the WNT5A or the Foxy-5 peptide to primarily a Frizzled receptor protein (Fz) followed by an intracellular signalling cascade in the cell eventually leading to reduction of checkpoint molecules such as PD-L1, CTLA4 and CD47. Thus, Wnt5A peptides including Foxy-5 are agonists which mimics the function of WNT5A and are thus not WNT pathway inhibitors.

The term surrounding non-cancer cells, as used herein, means morphologically normal cells, of the same tissue type from which the tumour has originated, enclosing or encircling the tumour tissue.

The term checkpoint in the context of the invention is defined as anyone of the proteins expressed by a tumour cell, a T-cell or a NK-cell. The protein expressed by the tumour cell is also sometimes specifically denoted as a checkpoint ligand, such that the specific protein is denoted with an “L” in the name, such as PD-L1.

The term checkpoint inhibitor in the context of the invention is defined as a molecule that binds specifically to a checkpoint protein expressed by any of a tumour cell, a T cell or NK-cell as defined above.

The term upregulated expression is to be understood as an increase in the quantity of a cellular component, such as CTLA-4 and/or PD-L1 in a cell, in response to an external stimulus such as treatment with WNT5A peptide or Foxy-5 as compared to a cell which has not been exposed to such external stimulus.

The term responsive to or sensitive to immune check point inhibitors is to be understood as subjects having checkpoints, preferably CTLA-4, PD-L1 and/or CD47 expression by any of tumour cells or infiltrating immune cells as well as their respective counterparts.

The term agonist is to be understood as a substance which initiates a physiological response when combined with a receptor as opposed to antagonist which is a substance which interferes with or inhibits the physiological action of another.

### EXAMPLES

### Example 1 (Protocol LEV 197)

Purpose: Analysis of the immune response against cancer antigen induced after tumour challenge and treatment with immunotherapy in BALB/c mice. Animals were purchased 6-8 weeks old from Envigo and allowed 1 week or more of rest after arrival before inclusion in experiments.

TABLE 1
Groups:

| | Mice | Vaccination scheme | Termination |
|---|---|---|---|
| A | 5 | Tumor, PBS | Tumor challenge (4T1- |
| | | | Luc) |
| B | 5 | Tumor, PD-L1, CTLA-4 | Tumor challenge (4T1- |
| | | | Luc) |
| C | 5 | Tumor, Foxy-5 | Tumor challenge (4T1- |
| | | | Luc) |
| D | 5 | Tumor, PD-L1, CTLA-4, | Tumor challenge (4T1- |
| | | Foxy-5 | Luc) |

Tumour Challenge: Day 0: 5*10\hat{ }4 4T1-Luc cells in 100 uL S.C.
Foxy-5 injection (i.p., 100 ul, 40 ug per mouse): Day 0, 4, 8, 12, 16->200 ug/mouse in total
When most tumour are palpable: injection of PD-L1 (BioXcell BE0146) and CTLA-4 (BioXcell BE0164) (i.p., 100 ul):
   1st injection: PD-L1-200 ug/CTLA4-200 ug
   2nd injection: PD-L1-200 ug/CTLA4-100 ug
   3rd injection: PD-L1-200 ug/CTLA4-100 ug
Mice was euthanized

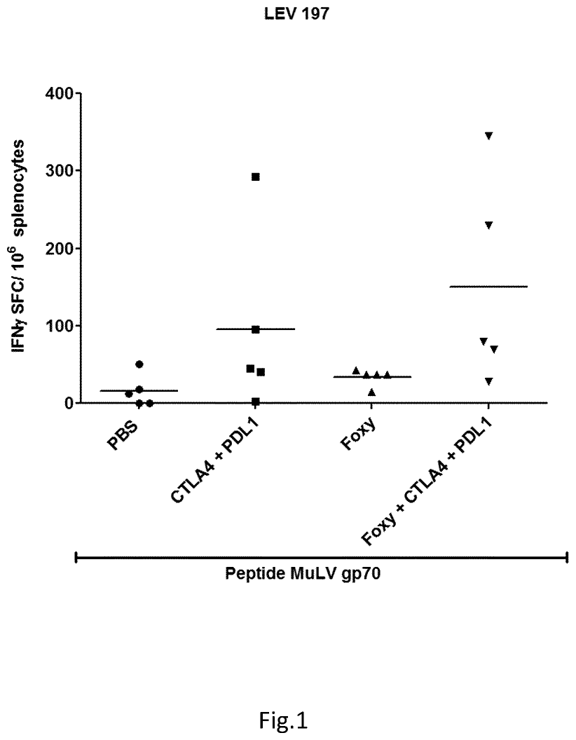

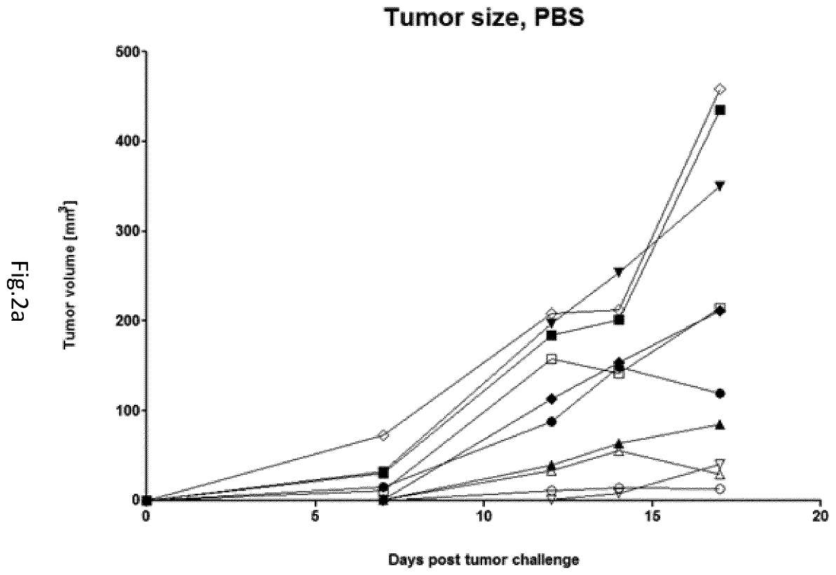

Result in FIG. 1. Conclusion: From subcutaneous tumours, 4T1 specific T cell responses with Foxy-5 and CTLA-4 or PD-L1 inhibitor co-treatment were directly observable ex-vivo. IFNγ spot forming cells were increased after MuLV gp70 peptide stimulation in cells from co-treated animals as compared to PBS control or single-treated animals.

### Example 2 (Protocol LEV 221)

Purpose: Analysis of the immune response against cancer antigen induced after tumour challenge and treatment with immunotherapy in BALB/c mice

TABLE 2
Groups:

| | | Vaccination | |
|---|---|---|---|
| | Mice | scheme | Termination |
| A | 10 | Tumor, PBS | Tumor challenge |
| | | | (4T1- Luc) |
| B | 10 | Tumor, PD-L1, | Tumor challenge |
| | | CTLA-4 | (4T1- Luc) |
| C | 10 | Tumor, Foxy-5 | Tumor challenge |
| | | | (4T1- Luc) |
| D | 10 | Tumor, PD-L1, | Tumor challenge |
| | | CTLA-4, Foxy-5 | (4T1- Luc) |

Tumour Challenge: Day 0: 5*10\hat{ }5 4T1 cells in 100 uL S.C.
Foxy-5 injection (i.p., 100 ul, 40 ug per mouse): Day 0, 4, 8, 12, 16->200 ug/mouse in total needed
injection of PD-L1 (BioXcell BE0146) and CTLA-4 (BioXcell BE0164) (i.p., 100 ul) on day 8, 12 and 16:
   1st injection: PD-L1-200 ug/CTLA4-200 ug
   2nd injection: PD-L1-200 ug/CTLA4-100 ug
   3rd injection: PD-L1-200 ug/CTLA4-100 ug
Mice euthanized on day 17.

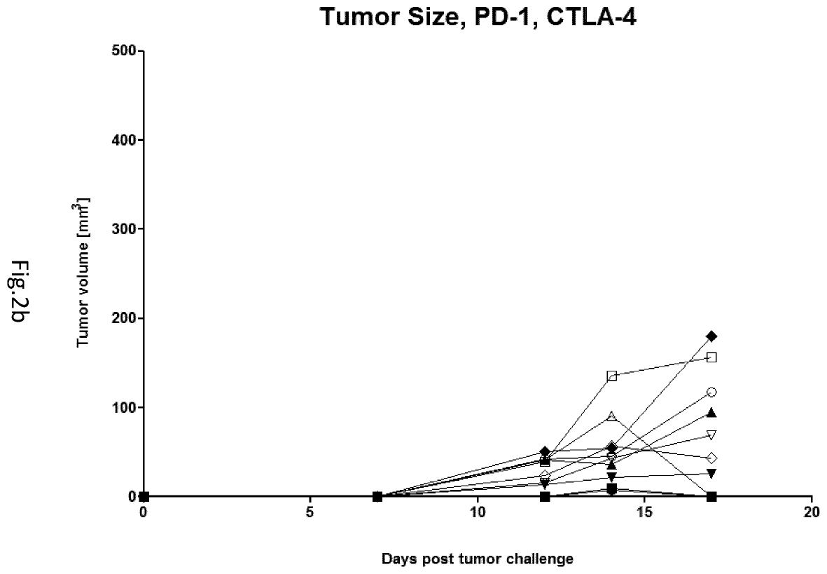

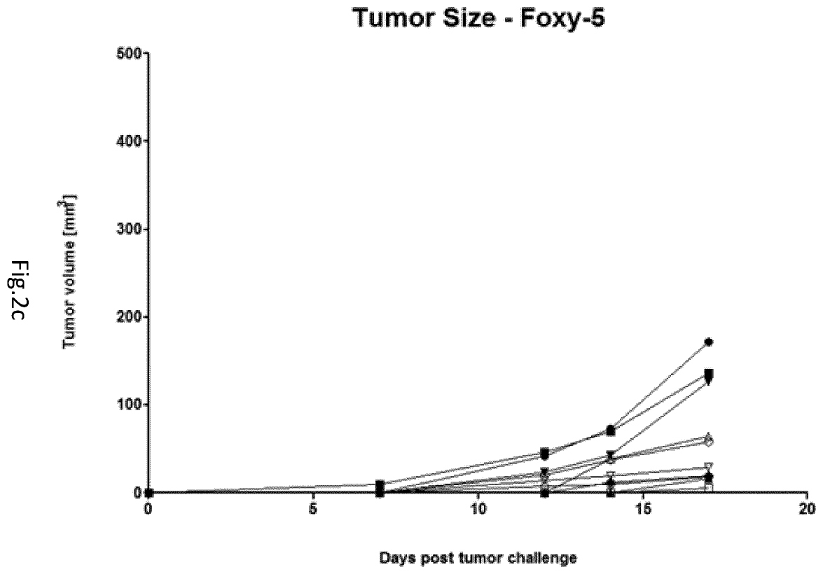

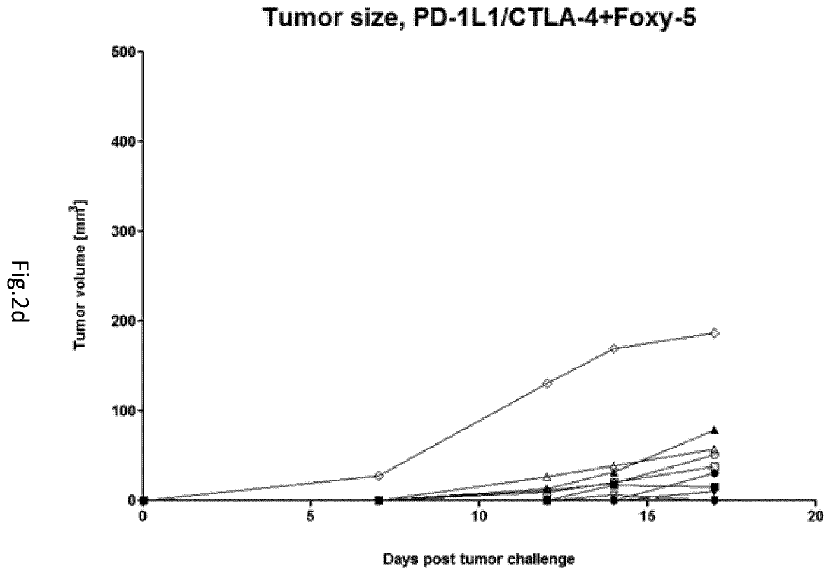

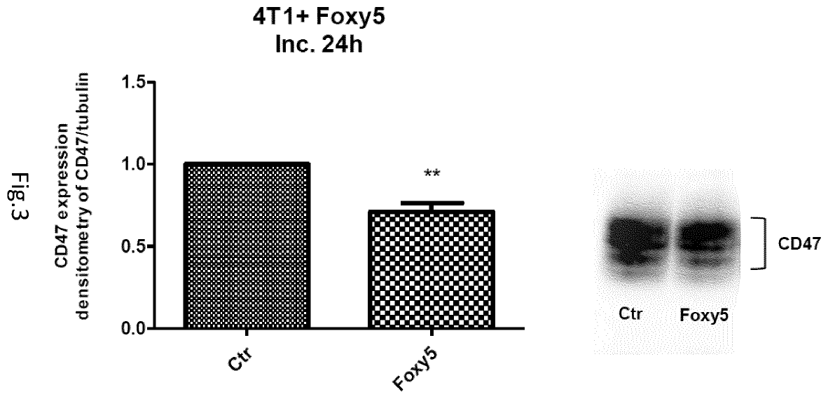

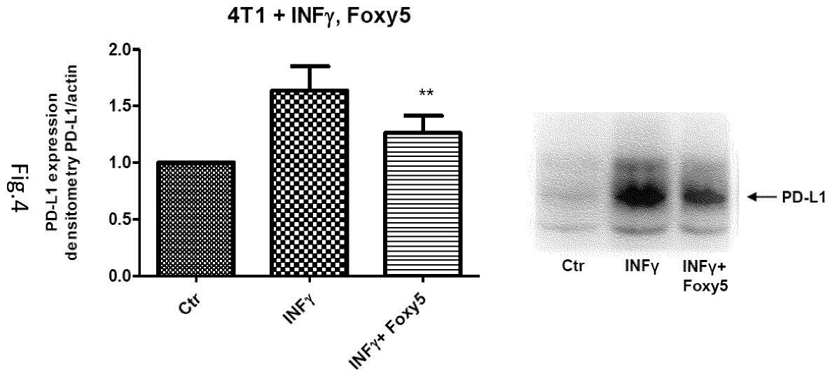

Result in FIGS. 2a-d. Conclusion: High-dose implantation of 4T1luc cells resulted in significantly reduced tumour growth (p<0.05 on last two measurements) in PD-L1- and CTLA-4-inhibitor treated groups, but most surprisingly beyond cumulative in the combined Foxy-5/ICI treated group.

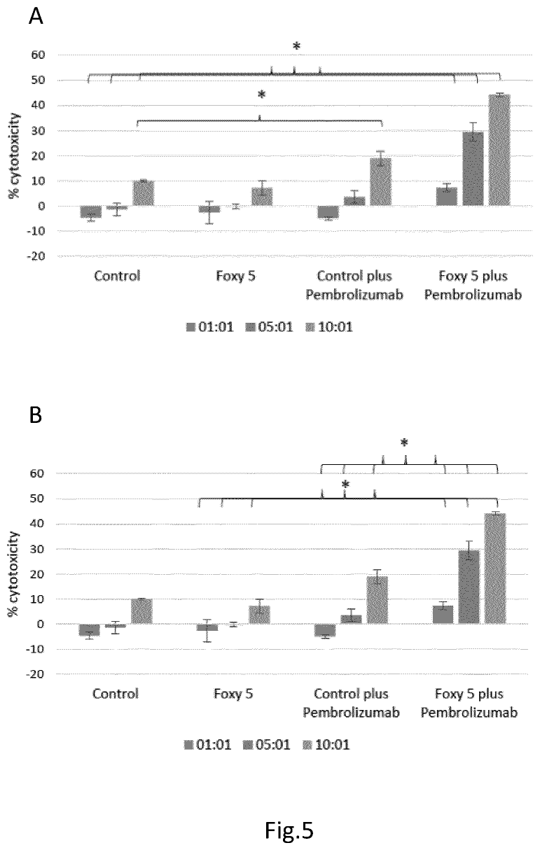

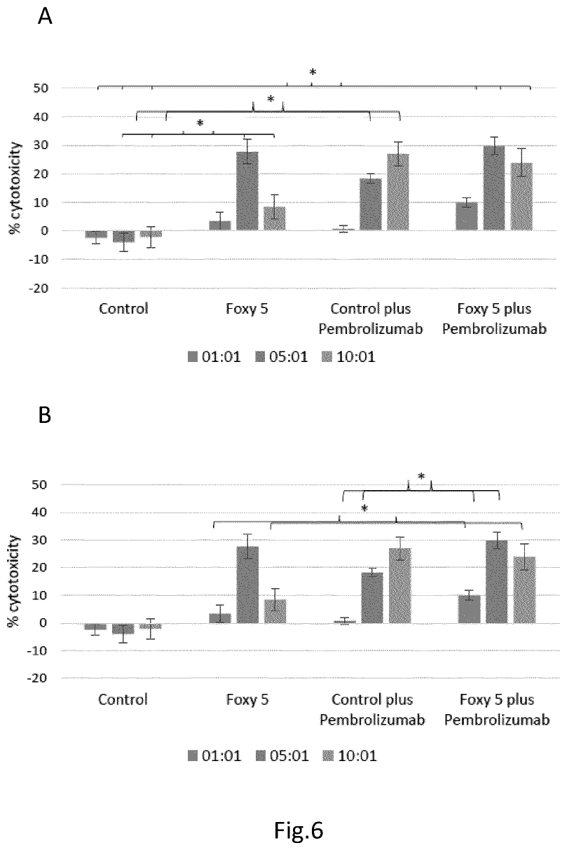

### Example 3 (FIGS. 3 and 4 Respectively)

Purpose: The ability of the WNT5A in this context, agonist, denoted, Foxy-5, to reduce the expression of PD-L1 and CD47 on the cell surface of breast- and colon cancer cells was examined. It is well established that the anti-phagocytosis cell surface molecule CD47, generating a “don't eat me signal” is over-expressed broadly among tumour types.

The investigation of the relationship between WNT5A signalling and CD47 expression was started in the triple-negative and WNT5A-negative breast cancer cell line 4T1. Results showed a substantial CD47 expression in these cells which is significantly reduced upon stimulation with Foxy-5 (24 h, n=4).

Next is was investigated how Foxy-5 possible would affect the expression of PDL1 in 4T1 cells. It was observed that non-stimulated 4T1 cells in tissue culture express a limited amount of PD-L1. Therefore, pre-stimulation (6 h) with Interferon gamma (IFNγ), a known inducer of PD-L1 in cancer cells that is present in the tumour microenvironment took place. Treatment of IFNγ pre-stimulated cells that had been “rested” overnight in the absence of any stimuli were then stimulated with Foxy-5 (24 h).

Conclusion: It is known that CD47 is an immune-suppressive checkpoint although it has not been explored as much as PD-1/PD-L1 as an immuno-therapeutic target in human cancer treatment. The results in FIG. 3 suggest Foxy-5 reduces CD47 expression thereby supporting its use in cancer treatment.

Foxy-5 treatment resulted in a significant reduced expression of PD-L1 in IFNγ-stimulated cells (FIG. 4, n=5). These results support a role for Foxy-5 in combination therapy, promoting existing treatments with checkpoint inhibitors by providing a reduction of PD-L1 expression to supplement PD-L1 blockade, and in particular by inhibiting CD47 which is known to act synergistically with PD-L1 blockade.

Reducing CD47 expression by a small molecule will be highly valuable as CD47 blockade requires extremely high doses to reach efficacious levels in humans.

### Example 4

Purpose: Examining the cytotoxic effect of Foxy-5 and an anti-PD-L1 antibody alone or in combination in a functional immune response assay with different breast cancer cell lines.

Method

Peripheral blood mononuclear cells (PBMCs) were isolated from whole blood using Ficoll Paque based density centrifugation.

SKBR3 (low in PD L1, denoted PD L1+) or HCC1954 (high in PD L1, denoted PD L1+++) cells were stained with 0.1 mM CFSE.

Effector cells (EC), i.e. PMMCs and target cells (TC), i.e. SKBR3 or HCC1954 cells, respectively, were brought to a concentration of 2×10 5 cells/ml.

Cells were treated with and without Foxy5 (100 μM), the cells were also treated with and without pembrolizumab (10 μg/μl).

Cells were plated at EC:TC ratios of 1:1 (first bar), 5:1 (second bar) and 10:1 (third bar) along with basal cell death controls and total cell death controls. Once plated, cells were spun down and incubated for 12 hours. After 12 hours cells were re suspended with 5 μg/ml 7AAD. Cells were then analysed on the Guava flow cytometer.

Based on staining patterns live/dead cell and immune/cancer cell differentiation can be determined and the direct cytotoxicity in terms of the cell death percentage was calculated.

Results in FIG. 5—SKBR3 Cell Line

A. SKBR3 Cytotoxicity Relative to Vehicle Control:

Direct cytotoxicity elicited by PBMCs against the SKBR3 cell line in the presence of FOXY-5 and/or pembrolizumab relative to vehicle control. 1:1 (first bar), 5:1 (second bar) and 10:1 (third bar) EC:TC ratios are shown. Error bars represent standard deviation of technical triplicate experiments. The Student's t test was used to determine statistical significance. * denotes p<0.05.

B. SKBR3 Combination Vs. Single Agents:

Direct cytotoxicity elicited by PBMCs against the SKBR3 cell line in the presence of FOXY-5 and pembrolizumab relative to either treatment alone. 1:1 (first bar), 5:1 (second bar) and 10:1 (third bar) EC:TC ratios are shown. Error bars represent standard deviation of technical triplicate experiments. The Student's t test was used to determine statistical significance. * denotes p<0.05.

Results in FIG. 6—HCC1954 Cell Line

A. HCC1954 Cytotoxicity Relative to Vehicle Control:

Direct cytotoxicity elicited by PBMCs against the HCC1954 cell line in the presence of FOXY-5 and/or pembrolizumab relative to vehicle control. 1:1 (first bar), 5:1 (second bar) and 10:1 (third bar) EC:TC ratios are shown. Error bars represent standard deviation of technical triplicate experiments. The Student's t test was used to determine statistical significance. * denotes p<0.05.

B. HCC1954—Combination Vs. Single Agents:

Direct cytotoxicity elicited by PBMCs against the SKBR3 cell line in the presence of FOXY and pembrolizumab relative to either treatment alone. 1:1 (first bar), 5:1 (second bar) and 10:1 (third bar) EC:TC ratios are shown. Error bars represent standard deviation of technical triplicate experiments. The Student's t test was used to determine statistical significance. * denotes p<0.05.

Further description of the results:

SKBR3 (PD L1 Low)

Relative to vehicle control: Foxy5 alone had no effect on direct PBMC cytotoxicity or trastuzumab mediated ADCC. Pembrolizumab alone increased direct PBMC cytotoxicity at the 10:1 ratio and decreased trastuzumab mediated ADCC at 10:1 and 5:1 ratios Foxy5+pembrolizumab increased direct PBMC cytotoxicity at all three ratios (1:1, 5:1 and 10:1).

Combination vs. single agents: The combination of Foxy5 and pembrolizumab increased direct cytotoxicity at all three ratios.

HCC1954 (PD L1 High)

Relative to vehicle control: Foxy5 alone increased direct PBMC cytotoxicity at 5:1 and 10:1 ratios. Pembrolizumab alone increased direct PBMC cytotoxicity at 5:1 and 10:1 ratios. Foxy5+pembrolizumab increased direct PBMC cytotoxicity at all three ratios, and decreased trastuzumab mediated ADCC at 5:1 and 10:1 ratios. Foxy5 increased overall cytotoxicity at 1:1 and 5:1 ratios when used alone and in combination with pembrolizumab.

Combination Vs. Single Agents

Foxy-5 increased direct cytotoxicity and overall cytotoxicity when added to pembrolizumab at ratios of 1:1 and 5:1. Pembrolizumab increased direct cytotoxicity when added to Foxy-5 at ratios of 1:1 and 10:1.

TABLE
p values for immune function assay:

| P-values | | SKBR3 | HCC1954 |
|---|---|---|---|
| Control vs. FOXY-5 | 1:1 | 0.489304852 | 0.05641 |
| | 5:1 | 0.494794119 | 0.000574* |
| | 10:1 | 0.180077069 | 0.030958* |
| Control vs. Pembro | 1:1 | 0.756736289 | 0.087276588 |
| | 5:1 | 0.069274026* | 0.000439071* |
| | 10:1 | 0.005657495* | 0.000837136* |
| Control vs. F + P | 1:1 | 0.000650842* | 0.001381* |
| | 5:1 | 0.000301788* | 0.000191* |
| | 10:1 | 2.43025E−07* | 0.001848* |
| FOXY-5 vs. F + P | 1:1 | 0.023303476* | 0.033724* |
| | 5:1 | 0.000187035* | 0.542002 |
| | 10:1 | 2.63607E−05* | 0.013869* |
| Pembro vs. F + P | 1:1 | 0.000261946* | 0.001637* |
| | 5:1 | 0.000556792* | 0.004274* |
| | 10:1 | 0.000126688* | 0.44592 |
| Pembro vs Foxy | 1:1 | 0.417375778* | 0.238849* |
| | 5:1 | 0.061224483* | 0.026208* |
| | 10:1 | 0.0073617* | 0.005388* |

*denotes p < 0.05
Pembrolizumab is also denoted Pembro or P, Foxy-5 is also denoted F

### CONCLUSION

In conclusion it is shown in the above experiments that the treatment of SKBR3 and HCC1954 cells with Foxy-5 in combination with a PD-L1 checkpoint inhibitor, Pembrolizumab is cytotoxic to the cancer cells and that the combination of the two drugs is more cytotoxic to the cells than when the cells are treated with the drugs separately.

1-15. (canceled)

16. A WNT5A peptide or derivatives thereof in combination with one or more checkpoint inhibitors for use in treatment of colon cancer, colorectal cancer or breast cancer in a subject in need thereof, the WNT5A peptide comprising X_{A}DGX_{B}EL (SEQ. ID. NO. 2), or a formylated derivative thereof, wherein X_{A} is methionine (M) or norleucine, X_{B} is cysteine (C) or alanine (A), wherein the total length of the peptide is equal to or less than 50 amino acids, wherein said peptide and said checkpoint inhibitor are either combined or separate and/or are administered either simultaneously or sequentially.

17. A WNT5A peptide or derivatives thereof in combination with one or more checkpoint inhibitors for use in treatment of colon cancer, colorectal cancer or breast cancer in a subject in need thereof according to claim 16, wherein said subject is defined as being responsive to immune checkpoint inhibitors.

18. The WNT5A peptide or derivatives thereof in combination with one or more checkpoint inhibitors for use in the treatment of colon cancer, colorectal cancer or breast cancer in a subject in need thereof according to claim 16, wherein the at least one checkpoint inhibitor is an inhibitor of an immune checkpoint molecule selected from the group consisting of CTLA-4, PD-1, PD-L1 and CD47.

19. The WNT5A peptide or derivatives thereof in combination with one or more checkpoint inhibitor for use in the treatment of colon cancer, colorectal cancer or breast cancer in a subject in need thereof according to claim 16, wherein the checkpoint inhibitor is an anti-CTLA4-antibody, anti-PD-1-antibody, anti-PD-L1-antibody and/or anti-CD47-antibody.

20. The WNT5A peptide or derivatives thereof in combination with one or more checkpoint inhibitor for use in the treatment of colon cancer, colorectal cancer or breast cancer in a subject in need thereof according to claim 19, wherein the anti-CTLA-4 antibody is ipilimumab or tremelimumab.

21. The WNT5A peptide or derivatives thereof in combination with one or more checkpoint inhibitor for use in the treatment of colon cancer, colorectal cancer or breast cancer in a subject in need thereof according to claim 19, wherein the anti-PD-L1-antibody antibody is atezolizumab, avelumab, durvalumab or pembrolizumab.

22. The WNT5A peptide or derivatives thereof in combination with one or more checkpoint inhibitor for use in the treatment of colon cancer, colorectal cancer or breast cancer in a subject in need thereof according to claim 16, wherein the dosage of checkpoint inhibitor used is reduced as compared to the dosage used when not administering WNT5A simultaneously or sequentially.

23. The WNT5A peptide or derivatives thereof in combination with one or more checkpoint inhibitor for use in the treatment of colon cancer, colorectal cancer or breast cancer in a subject in need thereof according to claim 16, wherein the subject in need thereof has an upregulated tumour expression of one or more immune checkpoint molecule selected from the group consisting of CTLA-4, PD-L1 and/or CD47.

24. The WNT5A peptide or derivatives thereof in combination with one or more checkpoint inhibitor for use in the treatment of colon cancer, colorectal cancer or breast cancer in a subject in need thereof according to claim 16, wherein the checkpoint inhibitor is an antiPD-L1-antibody and/or an anti-CTLA4-antibody and wherein the subject in need thereof has an upregulated tumour expression of CTLA-4, PD-L1 and/or CD47.

25. The WNT5A peptide or derivatives thereof in combination with one or more checkpoint inhibitor for use in the treatment of colon cancer, colorectal cancer or breast cancer in a subject in need thereof according to claim 16, wherein the WNT5A peptide is selected from the group consisting of:
(SEQ. ID. NO. 3)
MDGCEL,
(SEQ. ID. NO. 4)
GMDGCEL,
(SEQ. ID. NO. 5)
EGMDGCEL,
(SEQ. ID. NO. 6)
SEGMDGCEL,
(SEQ. ID. NO. 7)
TSEGMDGCEL,
(SEQ. ID. NO. 8)
KTSEGMDGCEL,
(SEQ. ID. NO. 9)
NKTSEGMDGCEL,
(SEQ. ID. NO. 10)
CNKTSEGMDGCEL,
(SEQ. ID. NO. 11)
LCNKTSEGMDGCEL,
(SEQ. ID. NO. 12)
RLCNKTSEGMDGCEL,
(SEQ. ID. NO. 13)
GRLCNKTSEGMDGCEL,
(SEQ. ID. NO. 14)
QGRLCNKTSEGMDGCEL,
(SEQ. ID. NO. 15)
TQGRLCNKTSEGMDGCEL,
(SEQ. ID. NO. 16)
GTQGRLCNKTSEGMDGCEL,
and
(SEQ. ID. NO. 17)
LGTQGRLCNKTSEGMDGCEL.

26. The WNT5A peptide in combination with one or more checkpoint inhibitor for use in the treatment of colon cancer, colorectal cancer or breast cancer in a subject in need thereof according to claim 16, wherein the WNT5A peptide is hexapeptide MDGCEL (SEQ. ID. NO. 3).

27. A method for treating colon cancer, colorectal cancer or breast cancer in a subject, said subject being defined as responsive to immune checkpoint inhibitors and said subject has an upregulated tumour expression of one or more immune checkpoint molecule selected from the group consisting of CTLA-4, PD-L1 and/or CD47, comprising administering a WNT5A peptide comprising X_{A}DGX_{B}EL (SEQ. ID. NO. 2), or a formylated derivative thereof, wherein X_{A} is methionine (M) or norleucine, X_{B} is cysteine (C) or alanine (A), wherein the total length of the peptide is equal to or less than 50 amino acids.

28. the method of claim 27, wherein the WNT5A peptide is selected from the group consisting of:
(SEQ. ID. NO. 3)
MDGCEL,
(SEQ. ID. NO. 4)
GMDGCEL,
(SEQ. ID. NO. 5)
EGMDGCEL,
(SEQ. ID. NO. 6)
SEGMDGCEL,
(SEQ. ID. NO. 7)
TSEGMDGCEL,
(SEQ. ID. NO. 8)
KTSEGMDGCEL,
(SEQ. ID. NO. 9)
NKTSEGMDGCEL,
(SEQ. ID. NO. 10)
CNKTSEGMDGCEL,
(SEQ. ID. NO. 11)
LCNKTSEGMDGCEL,
(SEQ. ID. NO. 12)
RLCNKTSEGMDGCEL,
(SEQ. ID. NO. 13)
GRLCNKTSEGMDGCEL,
(SEQ. ID. NO. 14)
QGRLCNKTSEGMDGCEL,
(SEQ. ID. NO. 15)
TQGRLCNKTSEGMDGCEL,
(SEQ. ID. NO. 16)
GTQGRLCNKTSEGMDGCEL,
and
(SEQ. ID. NO. 17)
LGTQGRLCNKTSEGMDGCEL.
